(19) United States
(12) Patent Application Publication
(10) Pub. No.: US 2012/0270292 A1
(43) Pub. Date: Oct. 25, 2012

(54) Photo-Inactivated Viruses and Systems and Methods of Using the Same

(75) Inventors: Julia Hilliard, Atlanta, GA (US); David Katz, Atlanta, GA (US)

(73) Assignee: GEORGIA STATE UNIVERSITY RESEARCH FOUNDATION, Atlanta, GA (US)

(21) Appl. No.: 13/518,026

(22) PCT Filed: Dec. 20, 2010
(86) PCT No.: PCT/US10/61335

§ 371 Date: Jun. 21, 2012

Related U.S. Application Data
(60) Provisional application No. 61/288,756, filed on Dec. 21, 2009.

(51) Int. Cl. C12N 13/00; C12N 1/20; C12N 1/14; C12N 7/04

(52) U.S. Cl. 435/173.3; 435/236; 435/252.1; 435/254.1; 435/173.1

(57) ABSTRACT

The present disclosure relates generally to systems and methods for the photo-inactivation of microorganisms. More specifically, the present invention is directed towards the photo-inactivation of microorganisms, such as viruses, using at least one furanocoumarin and broad spectrum pulsed light. For example, an aspect of the present invention includes a method for inactivating a herpesvirus, such as herpes B virus or herpes virus papio 2 using a psoralen and broad spectrum pulsed light.

### CROSS-REFERENCE TO RELATED APPLICATIONS

This application claims, under 35 U.S.C. §119(e), the benefit of U.S. Provisional Application Ser. No. 61/288,756, filed 21 Dec. 2009, the entire contents and substance of which are hereby incorporated by reference as if fully set forth below.

### BACKGROUND OF THE INVENTION

1. Technical Field

The various embodiments of the present disclosure relate generally to systems and methods for the photo-inactivation of microorganisms. More specifically, the various embodiment of the present invention are directed towards the photo-inactivation of microorganisms, such as viruses, using at least one furanocoumarin and broad spectrum pulsed light.

2. Description of Related Art

Herpes B virus (Herpesvirus simiae or Cercopithecine herpesvirus 1), a member of the Alphaherpesvirinae subfamily and the Simplexvirus group, is known to occur naturally in macaques (Macaca spp). Infection of macaques may be asymptomatic or may cause a mild disease. Infection of other species, such as humans, is rare but results in severe, and if untreated, lethal disease.

Past infections are determined by detection of anti B virus antibodies using serological assays. Serological diagnosis of B virus infections in humans, however, is complicated by the relatively high prevalence of the immunologically cross-reacting herpes simplex virus infections (e.g., HSV-1 and/or HSV-2). Past infections in macaques can be established without these complications because the only simplexvirus known to infect macaques is B virus. Identifying B virus infected macaques is important for managing macaques in captivity, for developing specific pathogen free colonies and for the prevention of the potential exposure and infection of humans who handle macaques.

Thus, what are needed are compositions, systems, and methods for the identification of individuals infected with a microorganism. The focus of the current application is to such novel composition, systems, and methods for the identification of individuals infected with a microorganism, such as B virus.

### BRIEF SUMMARY OF THE INVENTION

The various embodiments of the present disclosure relate generally to systems and methods for the photo-inactivation of microorganisms, and more particularly, to the photo-inactivation of viruses using at least one furanocoumarin and broad spectrum pulsed. For example, an aspect of the present invention comprises a method for inactivating a microorganism, comprising: providing at least one furanocoumarin to a microorganism; and exposing the microorganism to at least one pulse of a broad spectrum pulsed light, thereby inactivating the microorganism. The microorganism can be selected from the group consisting of viruses, bacteria, and fungi, and preferably comprises a virus. An exemplary virus comprises a herpesvirus, such as herpes B virus or herpes virus papio 2. The furanocoumarin can comprise a psoralen, and the psoralen can be used at a concentration ranging from about 0.1 μg/ml to about 60 μg/ml. In an exemplary embodiment, the psoralen is present in a concentration of at least about 5 μg/ml. Exposing the microorganism to at least one pulse of a broad spectrum pulsed light can comprise exposing the microorganism to about 0.45 Joule/cm² to about 13.5 Joules/cm² of broad spectrum light. In another embodiment, exposing the microorganism to at least one pulse of a broad spectrum pulsed light can comprise exposing the microorganism to at least about 4.05 Joules/cm² of broad spectrum light to about 13.5 Joules/cm² of broad spectrum light.

Another aspect of the present invention comprises an inactivated microorganism comprising a photo-chemically inactivated nucleic acid, wherein the photo-chemically inactivated nucleic acid is photo-chemically inactivated by at least one furanocoumarin and at least one pulse of a broad spectrum pulsed light. The microorganism can be selected from the group consisting of viruses, bacteria, and fungi, and is preferably a virus. An exemplary virus comprises a herpesvirus, such as herpes B virus or herpes virus papio 2. The furanocoumarin can comprise a psoralen, and the psoralen can be used at a concentration ranging from about 0.1 μg/ml to about 60 μg/ml. In an exemplary embodiment, the psoralen is present in a concentration of at least about 5 μg/ml. Photo-chemical inactivation of the virus can involve exposing the microorganism to at least one pulse of a broad spectrum pulsed light, which can utilize about 0.45 Joule/cm² to about 13.5 Joules/cm² of broad spectrum light. In an exemplary embodiment, photo-chemical inactivation of the virus can involve exposing the microorganism to at least about 4.05 Joules/cm² of broad spectrum light to about 13.5 Joules/cm² of broad spectrum light. For example, an inactivated microorganism can be inactivated by exposure to psoralen at a concentration of at least about 5 μg/ml and at least one pulse of a broad spectrum pulsed light that comprises at least about 4.05 Joules/cm² of broad spectrum light.

Yet another aspect of the present invention comprises a system for detecting an antibody in a subject, comprising: an antigen component, wherein the antigen is exposed to a furanocoumarin and at least one pulse of a broad spectrum pulsed light; and a reporter component that is capable of detecting a binding of an antibody of a subject to at least a portion of the antigen. The antigen can be selected from the group consisting of a virus, a bacterium, and a fungus, and preferably comprises a virus. In an exemplary embodiment, the viral antigen is a herpesvirus antigen, which can include, but is not limited to an antigen from herpes B virus or herpes virus papio 2. The furanocoumarin is a psoralen, and the broad spectrum pulsed light can comprise about 0.45 Joule/cm² to about 13.5 Joules/cm² of broad spectrum light. In one embodiment, the antigen component can further comprise an antigen disposed on a substrate. The reporter component can comprise, for example, a reporter antibody capable of binding at least a portion of the antibody capable of binding at least a portion of the antigen.

Still another aspect of the present invention comprises a method for immunizing a subject, comprising: inactivating an immunogenic microorganism comprising exposing to the immunogenic microorganism to a furanocoumarin and to at least one pulse of a broad spectrum pulsed light; and administering an effective amount of the immunogenic microorganism to a subject to produce an immune response. Such a method contemplates use of an inactivated immunogenic microorganism to immunize a subject. The immunogenic microorganism can include a virus, a bacterium, a fungus, or combinations thereof. In an exemplary embodiment, the immunogenic microorganism comprises a virus, preferably a herpesvirus, and more preferably a herpes B virus or herpes virus papio 2. The furanocoumarin can comprise psoralen, which can be present in a concentration of about 0.1 μg/ml to about 60 μg/ml. In an exemplary embodiment, psoralen is present in a concentration of at least about 5 μg/ml. Exposing the immunogen to a furanocoumarin and to at least one pulse of a broad spectrum pulsed light can comprise exposing the immunogen to about 0.45 Joule/cm² to about 13.5 Joules/cm² of broad spectrum light, and more specifically exposing the immunogen to at least about 4.05 Joules/cm² of broad spectrum light.

Another aspect of the present invention comprises an antibody having specific affinity for at least a portion of an antigen, wherein the antigen is derived from a microorganism that has been exposed to at least one furanocoumarin and at least one pulse of a broad spectrum pulsed light. The antigen can be derived from a microorganism, such as a virus, a bacterium, or a fungus. In exemplary embodiment, the microorganism is a virus, more specifically a herpesvirus, and even more specifically a herpes B virus or a herpes virus papio 2. The furanocoumarin can be a psoralen that is present in a concentration of about 0.1 μg/ml to about 20 μg/ml. In an exemplary embodiment, the psoralen is present in a concentration of at least about 5 μg/ml. The at least one pulse of a broad spectrum pulsed light can comprises about 4.05 Joules/cm² to about 13.5 Joules/cm² of broad spectrum light. In an exemplary embodiment, the at least one pulse of a broad spectrum pulsed light comprises about at least about 4.05 Joules/cm² of broad spectrum light. The antibody can be a polyclonal antibody or a fragment thereof or monoclonal antibody or a fragment thereof.

Yet another aspect of the present invention comprises an inactivated microorganism comprising an inactivated nucleic acid, wherein the inactivated microorganism retains its antigenicity. The microorganism can include viruses, bacteria, or fungi. In an exemplary embodiment, the inactivated microorganism is a virus, such as herpesvirus. In an exemplary embodiment, the inactivated microorganism comprises herpes B virus or herpes virus papio 2. Te inactivated nucleic acid of inactivated microorganism can include a crosslinked nucleic acid. The inactivated microorganism is capable of producing an immune response in a subject that is substantially similar to an immune response produced by a non-inactivated microorganism.

### BRIEF DESCRIPTION OF DRAWINGS

FIG. 1 illustrates PCR results for the different herpes virus papio 2 (HVP2) samples that were exposed to broad spectrum pulsed light (BSPL) in the presence (+ psoralen) and absence (no psoralen) of psoralen.

FIG. 2 graphically depicts the antigenicity of HVP2 samples that were treated with BSPL as compared to the live HVP2 preparation (HVP-2 Prep). In the legend of the graph “P” stands for BSPL pulses and the number indicates the number of pulses.

FIG. 3 graphically depicts the antigenicity of HVP2 samples that were treated with BSPL plus psoralen and compared to the live HVP2 preparation to which psoralen was added but not exposed to BSPL (HVP-2+Psor). In the legend of the graph “P” stands for BSPL pulses and the number indicates the number of pulses.

FIG. 4 illustrates the PCR inhibition results for the different HVP2 samples that were exposed to BSPL in the presence (+ psoralen) and absence (no psoralen) of psoralen.

FIG. 5 provides a dose response curve of psoralen versus the number of HVP2 plaques from the data in Table 3.

FIG. 6 demonstrates PCR inhibition of HVP2 DNA by different concentrations of psoralen and 9 pulses of BSPL.

FIG. 7 shows PCR inhibition results for B virus samples that were exposed to BSPL in the presence of psoralen.

FIG. 8 demonstrates the antigenicity of B virus samples that were photo-inactivated using psoralen plus BSPL. A standard rhesus anti-B virus serum was titrated on both the photo-inactivated antigens and on a standard “Tween/DOC” antigen (BV Ag). In the legend of the graph “P” stands for BSPL pulses and the number indicates the number of pulses, UN=uninfected, control antigen.

FIG. 9 illustrates amplification of extracted DNA using B virus specific gB primers.

FIG. 10 demonstrates the antigenicity of the inactivated B virus immunogen as tested by tELISA.

FIG. 11 graphically depicts titers of mouse sera from three mice that were immunized with B virus (BV) grown in 3T3 cells in microtiter wells that were coated with the original immunogen and an uninfected (UN) control prepared from 3T3 cells.

FIG. 12 graphically depicts titers of the same three mouse sera as in FIG. 11 in microtiter plate wells that were coated with B virus antigen grown in Vero cells and uninfected (UN) Vero cell controls.

FIG. 13 illustrates an embodiment of a design of a BV-Immuno Dip Strip.

FIG. 14 is a schematic representation of the well location numbers in the 96 deep well box for placing and incubating the dip-strips that are labeled with the corresponding numbers.

FIGS. 15A-B illustrates expected negative (A) and positive (B) reactions with the By-Immuno Dip Strips. Note the band at the third reaction site (UN) should always be colorless.

FIG. 16 is a schematic of nitrocellulose preparation.

FIG. 17 is a schematic of nitrocellulose-card preparation

FIG. 18 is a schematic of strip preparation from the nitrocellulose card.

FIG. 19 is an embodiment of the a BV-Immuno Dip Strip.

### DETAILED DESCRIPTION OF THE INVENTION

Various embodiments of the present invention are directed to photo-inactivated microorganisms and systems and methods of using the same. For example, one embodiment of the present invention includes a method for inactivating a microorganism, comprising: providing at least one furanocoumarin to a microorganism; and exposing the microorganism to at least one pulse of a broad spectrum pulsed light, thereby inactivating the microorganism.

As used herein, the term “microorganism” refers to many bacteria, viruses, fungi, and parasites. In an exemplary embodiment of the present invention, the microorganism is a virus, which can include, but is not limited to, adenoviridae, arenaviridae, filoviridae, bornaviridae, bunyaviridae, herpesviridae, orthomyxoviridae, polyomaviridae, papillomaviridae, paramyxoviridae, parvoviridae, picornaviridae, poxviridae, reoviridae, retroviridae, rhabdoviridae, togaviridae, hepadnaviridae, and bacteriophages. More specifically, a virus can include adenovirus 2, canine adenovirues, Pinchinde virus, Lassa virus, Turlock virus, California encephalitis virus, herpes simplex virus 1, herpes simplex virus 2, cytomegalovirus, pseudorabies virus, Epstein-Barr virus, varicella zoster virus, B virus (Macacine herpesvirus 1), herpesvirus papio 2 (Papiine herpesvirus 2), influenza virus, simian virus 40, human papilloma virus, measles virus, mumps virus, parainfluenza virus, poliovirus, coxsackievirus, echovirus, vaccinia virus, fowlpox virus, blue tongue virus, Colorado tick fever virus, rotavirus, human immuno-deficiency virus, Rous sarcoma virus, murine sarcoma virus, human T-cell leukemia virus, rhabies virus, vesticular stomatitis virus, Western equine encephalitis virus, West Nile virus, dengue virus, St. Louis encephalitis virus, hepatitis B virus, hepatitis C virus, lambdaphage, and Rickettsia, among others. In an exemplary embodiment of the present invention, the virus is Macacine herpesvirus 1 (also referred to as Cercopithecine herpes virus 1, herpesvirus simiae, herpes B virus, or B virus) or Papiine herpesvirus 2 (also referred to as Cercopithecine herpes virus 16, or herpes virus papio 2).

Inactivation of the microorganism refers to inhibition, interference, prevention, reduction, or alteration of replication or synthesis of nucleic acids, such as DNA, RNA, or combinations thereof. As used herein, the terms “preventing,” “interfering,” “reducing,” “altering,” or “inhibiting” refer to a difference in degree from a first state, such as an untreated state in a microorganism, to a second state, such as a treated state in a microorganisms. For example, in the absence of treatment with the methods or compositions of the present invention, nucleic acid replication or synthesis occurs at a first rate. If a microorganism is exposed to treatment with the methods or compositions of the present invention, nucleic acid replication or synthesis occurs at a second rate that is altered, lessened, or reduced from the first rate. The terms “preventing,” “interfering,” “inactivating,” “reducing,” “altering,” or “inhibiting” may be used interchangeably through this application and may refer to a partial reduction, substantial reduction, near-complete reduction, complete reduction, or absence of nucleic acid replication or synthesis. As used herein, the term “nucleic acid” can refer to a nucleotide, a nucleoside, a polynucleotide or portion thereof, a genome or portion thereof, a gene or portion thereof, an oligonucleotide, an aptamer, a transcript, DNA, RNA, or a DNA/RNA chimera, among others.

As used herein, the term “furanocoumarin” refers to a chemical substance containing a furan ring fused to a benzopyrone. Exemplary furanocoumarins comprise naturally-occurring psoralens or derivatives thereof, synthetic psoralens and derivatives thereof, as well as combinations thereof. For example, a psoralen can be a methoxypsoralen (e.g., 8-MOP, 5-MOP), a trimethylpsoralen (TMP), a 4-aminomethyl-trioxsalen (AMT), or combinations thereof.

Providing at least one furanocoumarin to a microorganism comprises administering an effective amount of at least one furanocoumarin to intercalate a nucleic acid of the microorganism. The precise effective amount is an amount of the furanocoumarin composition that will yield effective results in terms of inactivation of a microorganism. This amount (i.e., dosage) may vary depending upon a number of factors, including, but not limited to, the characteristics of the furanocoumarin or derivative thereof, the microorganism, and the amount of broad spectrum pulsed light administered. For example, an effective amount of psoralen can have a concentration ranging from about 0.1 μg/ml to about 60 μg/ml. In one embodiment of the present invention, psoralen is used in a concentration greater than about 0.3 μg/ml. In another embodiment of the present invention, psoralen is used in a concentration of at least about 5 μg/ml. In yet another embodiment of the present invention, psoralen is used in a concentration of at least about 20 μg/ml. In still another embodiment of the present invention, psoralen is used in a concentration of at least about 50 μg/ml.

Exposing the microorganism to at least one pulse of a broad spectrum pulsed light can involve exposing a microorganism to one pulse of light or a plurality of pulses of light. A pulse of light is an amount of light that continues for a very short, but measurable time, for example, microseconds (μs). The number of pulses of light required to inactivate a microorganism may vary depending upon a number of factors, including, but not limited to, the characteristics and concentration of the furnaocoumarin or derivative thereof, the microorganism and its concentration, the light transparency of the medium in which the microorganism is suspended, the light transparency of the container that accommodates the microorganism suspension, and the source/wave length of the broad spectrum pulsed light, among others. In an exemplary embodiment of the present invention, the source of the broad spectrum pulsed light is a xenon lamp capable of generating a continuous broad-spectrum of light, ranging from about the deep UV spectrum through about the infrared spectrum. Ultraviolet (UV) light is electromagnetic radiation with a wavelength shorter than that of visible light, but longer than x-rays, in the range 10 nm to 400 nm, and energies from 3 eV to 124 eV (one eV is equivalent to 1.60217653×10⁻¹⁹ Joules.). Infrared (IR) radiation is electromagnetic radiation with a wavelength between 0.7 and 300 micrometer (μm), which equates to a frequency range between approximately 1 and 430 THz. Thus, broad-spectrum light can include wavelengths from about 10 nm to about 300 μm.

In an exemplary embodiment of the present invention, exposing the microorganism to at least one pulse of a broad spectrum pulsed light comprises exposing the microorganism to about 0.45 Joule/cm² to about 13.5 Joules/cm² of broad spectrum light. In one embodiment, a microorganism, such as herpes B virus, is exposed to about 5.4 Joules/cm² of broad spectrum light. For example, a microorganism, such as herpes B virus, is exposed to a cumulative amount of about 5.4 Joules/cm² of broad spectrum light. In another embodiment, a microorganism, such as herpes B virus, is exposed to about 12.15 Joules/cm² of broad spectrum light. For example, a microorganism, such as herpes B virus, is exposed to a cumulative amount of about 12.15 Joules/cm² of broad spectrum light. The single-dose amount or multiple-dose amount (in either case, the cumulative/total amount) of broad spectrum light for use in the present method typically ranges from about 4.05 Joules/cm² to about 13.5 Joules/cm², but can exceed this amount as long as the immunogenicity of the antigens is maintained. The cumulative amount of broad spectrum light may be delivered in pulses of various lengths, which can be separated by various lengths of time. For example, broad spectrum light can be delivered in a pulse width of about 360 μs, where three pulses can be generated per second with each pulse generating an energy of about 0.45 joules/cm² per pulse. Using such an example, a microorganism, such as herpes B virus, can be inactivated using about 12 pulses. In addition, the energy of each pulse may also be varied. The energies of each pulse can range from about 0.3 Joules/cm² per pulse to about 0.6 Joules/cm² per pulse. For example, a pulse with an energy of about 0.45 Joules/cm²/pulse can be used. Other pulse widths can also be used. For example, pulse widths from about 250 μs to about 450 μs can be used, and the number of pulses adjusted to obtain a cumulative amount of from about 4.05 Joules/cm² to about 13.5 Joules/cm², or in a more specific example, from about 3 Joules/cm² to about 13.5 Joules/cm^{2.}

Another aspect of the present invention includes an inactivated microorganism, comprising a photo-chemically inactivated nucleic acid, wherein the photo-chemically inactivated nucleic acid is photo-chemically inactivated by at least one furanocoumarin and at least one pulse of a broad spectrum pulsed light. The microorganism can be any of the microorganisms disclosed herein, and can be produced by any of the inactivation methods described herein. In an exemplary embodiment, the microorganism can be a virus, such as a herpesvirus, and more specifically, a Macacine herpesvirus 1 or Papiine herpesvirus 2 (HVP2). The inactivated microorganism of the present invention has enhanced function because of the combined characteristics of having inactivated DNA and retaining the structural integrity of its surface antigens. For example, inactivation of viruses by detergents is more effective for enveloped viruses then for non-enveloped viruses. Detergents disrupt lipid membranes of cell membranes and enveloped viruses by interacting with lipids and releasing proteins or glycoproteins from the lipid-rich envelopes. In an example of herpesviruses, a combination of surfactants (e.g., Tween 40) and a detergent (e.g., sodium deoxycholate) can be used to disrupt the envelope and thereby inactivate a herpesvirus. Using a detergent-based method, the DNA of the virus is not affected by this procedure. In contrast, the psoralen/BSPL technique described above damages nucleic acids (e.g., DNA and RNA) and therefore is not restricted to any particular microorganism, enveloped or not.

An inactivated microorganism can be used in a system for detecting an antibody. An antibody may be polyclonal or monoclonal, and may include fragments such as Fab, FC, heavy chains, light chains, constant, variable, or hypervariable fragments or regions, and any type of antibody including but not limited to IgM, IgG, IgA, IgD, and IgE. An antibody has specificity for at least a portion of an antigen. The phrase “having specificity for an antigen” with respect to the antibody as used herein can also be referred to as the “binding activity,” “binding affinity,” or “specific affinity” of the antibody relative to the target. These phrases may be used interchangeably herein and are meant to refer to the tendency of a ligand to bind or not to bind to a target. The energetics of these interactions are significant in “binding activity” and “binding affinity” because they define the necessary concentrations of interacting biomolecules, the rates at which these biomolecules are capable of associating, and the relative concentrations of bound and free biomolecules in a solution. The energetics are characterized through, among other ways, the determination of a dissociation constant, K_{d}. The specificity of the binding is defined in terms of the comparative dissociation constants (K_{d}) of the ligand for target as compared to the dissociation constant with respect to the ligand and other materials in the cellular environment or unrelated molecules in general. Typically, the K_{d} for an antibody with respect to the antigen will be at least 2-fold, preferably 5-fold, and more preferably 10-fold less than K_{d} with respect to target and the unrelated material or accompanying material in the cellular environment. Even more preferably, the K_{d} will be 50-fold less, more preferably 100-fold less, and more preferably 200-fold less than K_{d} with respect to target and the unrelated material or accompanying material in the cellular environment.

Such a system for detecting an antibody can comprise: an antigen component, wherein the antigen is exposed to a furanocoumarin and at least one pulse of a broad spectrum pulsed light; and a reporter component that is capable of detecting a binding of an antibody of a subject to at least a portion of the antigen. For example, the antigen component can be Macacine herpesvirus 1 or antigenic components thereof or Papiine herpesvirus 2 or antigenic components thereof. The furanocoumarin and pulsed light exposure can be in accordance with any of the methods described herein. The antigen component can be disposed on a substrate, such as for example, a microtiter plate, a nitrocellulose membrane, or the like. The reporter component can be a reporter antibody capable of binding at least a portion of the antibody capable of binding at least a portion of the antigen. Thus, the inactivated microorganism or component derived therefrom can be used to detect the presence of an antibody in a subject that has at least some specificity for the inactivated microorganism or component derived therefrom. For example, the system can be used to detect antibodies specific for Macacine herpesvirus 1 or Papiine herpesvirus 2 in a subject, such as a human or non-human primate.

An antigen that is exposed to a furanocoumarin and at least one pulse of a broad spectrum pulsed light can be used in many immunoassays, including, but not limited to enzyme-linked immunosorbent assay (ELISA), radioimmunoassay (RIA), magnetic immunoassays, immunoblotting (i.e., Western blotting), immunoprecipitation, immunohistochemistry, affinity chromatography, and flow cytometry, among others.

Another aspect of the present invention involves a method for immunizing a subject, comprising: inactivating an immunogenic microorganism comprising exposing the immunogenic microorganism to a furanocoumarin and to at least one pulse of a broad spectrum pulsed light; and administering an effective amount of the immunogenic microorganism to a subject to produce an immune response. Thus, the inactivated microorganisms of the present invention can be used to generate an immune response in a subject, such as an adaptive immune response or a innate immune response. In an exemplary embodiment of the present invention, the inactivated microorganism can elicit a B cell response, a T cell response, or a combination thereof. In another exemplary embodiment of the present invention, the inactivated microorganism can elicit a protective immune response. For example, the inactivated microorganisms of the present invention can be used to vaccinate a subject against a microorganism, such as virus. In one embodiment, the inactivated microorganism can be an inactivated herpes B virus that can be used to vaccinate a human or non-primate.

Using the inactivated microorganisms of the present invention, an antibody can be raised to the inactivated microorganism, where the antibody has a specific affinity for at least a portion of the inactivated microorganism. The antibody can be raised against an antigen derived from a microorganism selected from the group consisting of a virus, a bacterium, and a fungus. Such an antibody can be a polyclonal antibody or a monoclonal antibody, among others as discussed above. For example, using an inactivated herpes B virus, a polyclonal antibody can be raised to one or more epitopes of herpes B virus. In addition, a monoclonal antibody can be raised that has specificity to one of the one or more epitopes of herpes B virus. The antibody can be used in many immunoassays, including, but not limited to enzyme-linked immunosorbent assay (ELISA), radioimmunoassay (RIA), magnetic immunoassays, immunoblotting (i.e., Western blotting), immunoprecipitation, immunohistochemistry, affinity chromatography, and flow cytometry, among others.

Another aspect of the present invention includes an inactivated microorganism comprising an inactivated nucleic acid, wherein the inactivated microorganism retains its antigenicity. The phrase “retains its antigenicity” refers to the ability of an inactivated microorganism to bind to an antibody that is produced by an immune response to the live microorganism that is not treated by the systems and methods of the present invention. For example, in the case of antibody binding, an inactivated microorganism of the present invention would be recognized by at least a majority of the same antibodies that recognize a live microorganism at substantially the same titers.

Another aspect of the present invention includes an inactivated microorganism comprising an inactivated nucleic acid, wherein the inactivated microorganism retains its immunogenicity. The phrase “retains its immunogenicity” refers to the ability of an inactivated microorganism to produce an immune response in a subject that is substantially similar to an immune response produced by a live microorganism that is not treated by the systems and methods of the present invention.

All patents, patent applications, and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure. Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

### EXAMPLES

### Example 1

### A Modified Photoinactivation Technique for Inactivation of Viruses Using Psoralen and Broad Spectrum Light Pulses

Previous experimentation to photo-inactivate a virus involve a procedure in which a “black light lamp” (UVA) was used to irradiate HVP2-psoralen mixtures in Petri dishes. In this procedure, virus-psoralen mixtures were exposed to a “black light” UVA lamp for two sets of 30 min exposures. The results of this experimentation was unsatisfactory because it was time consuming, caused heating and evaporation, and most importantly resulted in poor antigenicity.

In an attempt to overcome the shortcomings of the above procedure, this example describes a modified psoralen photo-inactivation technique in which the photo-activation of the psoralen is done by using the SteriPulse-XL irradiation device (Model RS-3000C) from Xenon Corporation (Woburn, Mass.). Information regarding the SteriPulse-XL system is described in Xenon's publication, entitled “Sterilization & Decontamination using High Energy UV Light,” which is hereby incorporated by reference.

The SteriPulse-XL system employs a xenon lamp that generates broad spectrum pulsed light (BSPL) in short controlled pulses (360 microseconds per pulse (μs/pulse)). The intensity of the BSPL is approximately 50,000 to 100,000 times the energy level of the sun. BSPL comprises at least a UVA wavelength that facilitates the photo-activation of the psoralen, as well as UVB and UVC, which are associated with having germicidal properties.

Psoralens, which belong to the class of molecules known as furanocoumarins, intercalate nucleic acids. In the presence of UVA, psoralens alkylate nucleic acids to generate monoadducts and cross-links. For example, in the case of double-stranded DNA, psoralens alkylate nucleic acids at the 5,6-double bond of thymidines, effectively crosslinking the DNA duplex. This prevents DNA strand separation during transcription and replication.

Materials and Methods

Preparation of virus and antigen stocks in Vero cells. Vero cells (ATCC #CCL-81) were grown in 980 cm² roller bottles to 95% confluency, and subsequently infected (multiplicity of infection (MOI)=5) with HVP2 or B virus and maintained in Dulbecco's Modified Eagles Medium (DMEM) with high glucose supplemented with 1% fetal bovine serum (FBS) and antibiotics (penicillin and streptomycin). The infected cells were incubated for 22 to 24 hrs at 34° C. until cytopathic effect (CPE) can be observed. The cells were then scraped into the medium and centrifuged at 1500 rpm (514×g) for 10 min. Cell pellets were resuspended in 4.5 ml of sterile ultrapure water. The suspension was then treated by 3 cycles of freezing on dry ice and thawing in a 37° C. water bath. Each freezing cycle lasted for at least 15 min. Cell debris was removed by centrifugation (1500 rpm for 10 min) and the supernatant (about 5 ml) was saved. The virus titer of the preparation as determined by the standard plaque assay in Vero cells was approximately 10¹⁰ PFU/ml. Although cells in this embodiment were lysed through three cycles of freezing and thawing, cells can also be lysed by way of sonication. In some viral preparations, such as B virus, antigenic yield may be enhanced by using sonication-based methods.

Preparation of viral antigens from infected cells by detergent solubilization. Vero cells (ATCC #CCL-81) were grown in 980 cm² roller bottles to 95% confluency, infected (MOI=5) with HVP2 or B virus, and maintained in Dulbecco's Modified Eagles Medium (DMEM) with high glucose supplemented with 1% fetal bovine serum (FBS) and antibiotics (penicillin and streptomycin). The infected cells were incubated for 22 to 24 hrs at 34° C. until CPE can be observed. The cells were then scraped into the medium and centrifuged in 50 ml conical tubes at 1500 rpm (514×g) for 10 min. The supernatant was discarded, and the pellet was resuspended in 1.5 ml of water containing 2× Complete Protease Inhibitor (Roche). The final volume was adjusted to 2.0 ml with water containing 2× Complete Protease Inhibitor. The resuspended pellet was then treated with a final concentration of 1% Tween 40 (250 μl of 10% Tween 40) and 1% Sodium Deoxycholate (250 μl of 10% Sodium Deoxycholate) by adding one after the other with extensive mixing (vortexing) intervals.

Assessment of virus inactivation using a standard plaque assay in Vero cells. Assessment of virus inactivation was accomplished by either one of the two following techniques utilizing a standard plaque assay in Vero cells. 1. Vero cell monolayers grown in 24-well plates were infected with virus preparations for 48 or 72 hrs. If no cytopathic effect (CPE) was observed microscopically, cells from each well were scraped into a small amount of medium (500 μl), and replated on a new Vero-cell monolayer (in a 24-well plate) and incubated for another 72 hrs at 37° C. Cells were then fixed with 100% methanol and stained with crystal violet to facilitate the counting of plaques. 2. Vero cell monolayers in 24-well plates were infected with virus preparations for six days and inspected daily for the development of CPE. At the end of the incubation periods, cell monolayers were then fixed with 100% methanol and stained with crystal violet to facilitate CPE determination or counting of virus induced plaques. Absence of CPE or plaques by any of the techniques indicates absence of virus replication due to inactivation.

Assessment of virus antigenicity by tELISA. Virus antigenicity was assessed by tELISA, an assay for antibody detection and quantitation that is performed in 96-well microtitration-plates. The tELISA was performed essentially as previously described with some modifications in Katz D, Hilliard J K, Eberle R, Lipper S L (1986a). Briefly, herpesvirus antigens were adsorbed to microplate-wells by incubation for 20 min on a shaker at room temperature, or overnight at 4° C. After blocking with Blotto (3% skim milk) (1 h at 37° C.), adsorbed antigens were reacted (1 h at 37° C.) with serial dilutions of homologous standard antiserum pools. Anti human-IgG-alkaline phosphatase conjugate was then added and incubated (1 h at 37° C.) for the detection of antigen-bound monkey antibodies. After each of the incubation steps, microplates were washed 3 times with phosphate buffered saline (PBS) supplemented with 0.05% Tween 20 (PBST). The substrate, dinitro-phenyl phosphate, was added and incubated for 30 min at room temperature. Color intensity in optical density (OD) units was read in a micro-ELISA reader at a wavelength of 405 nm.

Assessment of DNA damage by Polymerase chain Reaction (PCR). DNA damage was demonstrated by the inhibition of a PCR amplification product formation, validating virus inactivation. The PCR primers were initially designed based on the gB gene sequence of B virus and the gL gene sequence of HVP2. The BV gB primer set amplifies a 1.3 kb fragment of B virus, while the HVP2 gL primer set amplifies a 1.2 kb fragment of HVP2. In later experiments, the following primers were used for amplification of both BV and HVP2 DNA. Primers were designed based on the BV gB sequence. The forward primer was 5′-GTGTACATGTCGCCGTTCTA-3′ (position 53972 in the BV genome). The reverse primer was 5′-GTGTACATGTCGCCGTTCTA-3′ (position 52659 in the BV genome). The amplimer expected if PCR occurred (i.e., if there was no damage to DNA) would be 1313 bp. The PCR was performed by using the PCR HotStar Kit (Qiagen) and 3 μl of purified DNA in 20 μl volume. The amplification was performed in an ABI Thermocycler 9600 using the following cycling conditions: 15 min 95° C., 35 two step cycles of 20 seconds at 95° C., and 40 seconds at 65° C. Then, the PCR products were run on 1% agarose gel along with a DNA marker to determine the presence of the PCR fragment of the expected size. The absence of the PCR fragment after amplification of a sample verified DNA damage.

Results

Comparison of HVP2 inactivation procedures using BSPL and the combined Psoralen-BSPL Inactivation Technique.

Experiment 1. The purpose of this experiment was to compare the inactivation procedure by BSPL to the photo-inactivation procedure in which a combination of psoralen and BSPL was used. Inactivation by BSPL alone was performed as follows: five 1 ml portions of a diluted HVP2 preparation (10⁸ PFU/ml) were transferred into 5 polyethylene tubing (Polytubing, 1″×1,500′ 2 Mil, Catalog #S-3520, ULINE, Atlanta Ga.) that were heat sealed at one end. The ends were then heat-sealed at a distance of 5 cm from the first seal. Another 5 ml of the diluted virus were first mixed with psoralen (4-Aminomethyl-trioxalen hydrochloride, Sigma, Catalogue #A4330 5 mg) to a final concentration of 20 μg/ml of psoralen and then transferred in 1 ml portions to 5 polyethylene tubings as described above. Each of the tubings (with and without psoralen) was irradiated with a different dose of BSPL. Pairs of polyethylene tubings from each group, one that contains the virus and the other that contains the virus and psoralen, were placed in a plastic tray, flat on a bed of crushed ice. The plastic tray was placed in the Steripulse chamber on at a distance of 8 shelves (4.26 inches) from the lamp window at the top of the chamber to achieve 0.45 Joules/cm² per pulse. Each of the tubings was exposed to 3, 6, 9, 12, and 15 pulses. The total energy was determined by multiplying the energy output per pulse (0.45 joule/cm²) by the number of pulses (See Table 1). Each of the samples was then tested for infectivity by the plaque assay. Table 1 provides the plaque assay results for the inactivation of HVP2 infectivity by BSPL compared to the photo-inactivation procedure in which a combination of psoralen and BSPL was used. The number of plaques for each BSPL dose are shown in bold numbers. While a minimum dose of 6 BSPL pulses were sufficient to inactivate the virus in the presence of psoralen, the number of pulses necessary to inactivate the virus by BSPL only was 12.

TABLE 1

| # of Pulses | 3 | 6 | 9 | 12 | 15 |
|---|---|---|---|---|---|
| Joules/cm² | 1.35 | 2.70 | 4.05 | 5.40 | 6.75 |
| BSPL + Psoralen | 2 | 0 | 0 | 0 | 0 |
| BSPL only | 100 | 8 | 2 | 0 | 0 |

The DNA damage that was caused by exposure of the HVP2 samples to BSPL or BSPL+psoralen was assessed by PCR. FIG. 1 shows that BSPL+psoralen inhibits the PCR at 9 pulses or higher. Exposure to BSPL alone causes a dose related decrease of band intensity, but a band can be clearly seen after an exposure of 12 BSPL pulses. Both the infectivity assays and PCR show the additive inactivation potency of psoralen. In this experiment, the PCR inhibition assay was a more sensitive assay, since amplification of DNA was still present in samples that were negative by the plaque assay. However, in other experiments (shown below), live virus was present in preparations that showed DNA damage by inhibition by PCR.

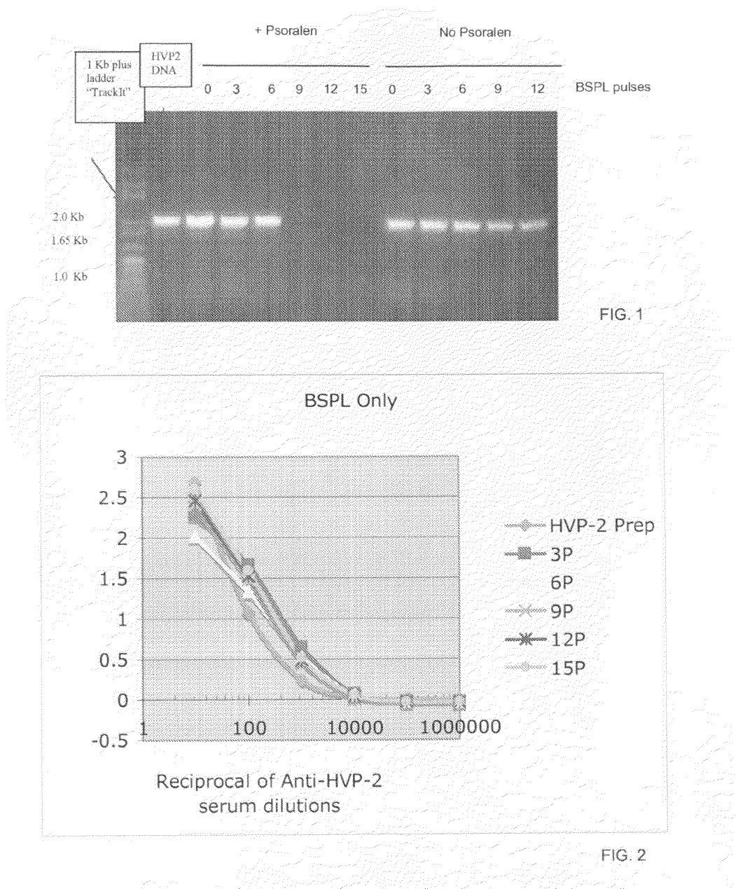

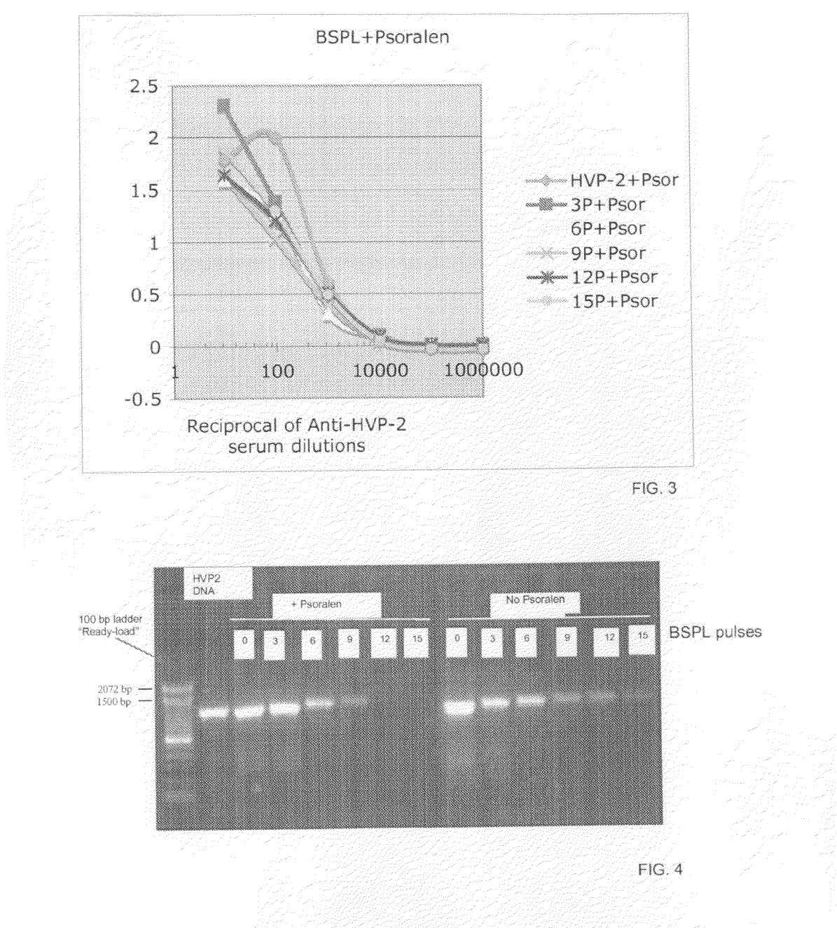

The different samples were each tested for antigenicity by tELISA. A 1:30 dilution of each sample was adsorbed to wells, washed, blocked with Blotto and used for the titration of an anti-HVP2 standard baboon serum. As can be seen in FIG. 2 for samples that were treated with BSPL and in FIG. 3 for samples treated with BSPL and psoralen, none of the treatments altered antigenicity of inactivated virus.

Experiment 2. The procedures described for Experiment 1 were repeated in this experiment with some minor variations. The samples exposed to 3 pulses of BSPL were not tested by the plaque assay. The plaque assay was performed for samples that were exposed to 6 or more BSPL pulses. Table 2 provides plaque assay results for the inactivation of HVP2 infectivity by BSPL compared to the photo-inactivation procedure in which a combination of psoralen and BSPL was used. The number of plaques for each BSPL dose are shown in bold numbers. All samples exposed to 3 BSPL pulses or higher were tested by PCR (FIG. 4). As shown in Table 2, the samples that were mixed with psoralen and exposed to 6 BSPL pulses or higher did not produce plaques. The samples that were exposed to BSPL only did produce plaques after exposure to 6, 9, and 12, but not after 15 pulses.

TABLE 2

| # of Pulses | 6 | 9 | 12 | 15 |
|---|---|---|---|---|
| Joules/cm² | 2.70 | 4.05 | 5.40 | 6.75 |
| BSPL + Psoralen | 0 | 0 | 0 | 0 |
| BSPL only | 48 | 4 | 2 | 0 |

The PCR results depicted in FIG. 4 were similar to those obtained in the first experiment, except that a weak DNA band could be seen after treatment with psoralen and exposure to 9 BSPL pulses. No bands were seen in psoralen treated samples that were exposed to 12 and 15 BSPL pulses. All samples that were exposed to BSPL only (3 to 15 pulses), showed DNA bands. The intensity of the bands decreased with the increase of the number of BSPL pulses.

### Effect of Psoralen Concentration on the Photo-Inactivation of HVP2.

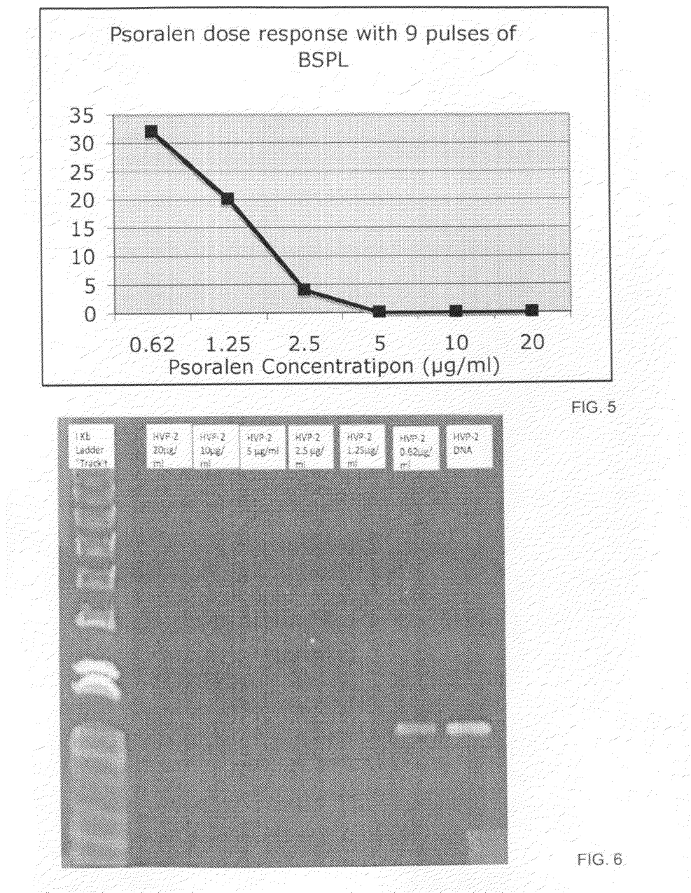

One ml portions of a HVP2 stock diluted to 10⁸ pfu/ml were prepared as for the previous experiment, mixed with different concentrations of psoralen (Table 3) and exposed to 9 pulses of BSPL as described above. Samples from each treatment were tested by the plaque assay (Table 3 and FIG. 5) and by PCR (FIG. 6). Table 3 provides the plaque assay results that demonstrate inactivation of HVP2 infectivity by different concentrations of psoralen and 9 pulses of BSPL. A concentration of 5.0 μg/ml of psoralen or higher plus 9 BSPL pulses inactivated the infectivity of the virus. Increasing number of plaques were observed with decreasing concentrations of psoralen (Table 3 and FIG. 5).

TABLE 3

| | Psoralen Concentration (μg/ml) | | | | | |
|---|---|---|---|---|---|---|
| | 20.0 | 10.0 | 5.0 | 2.5 | 1.25 | 0.62 |
| Number of plaques. | 0 | 0 | 0 | 4 | 20 | 32 |

The PCR indicates that as low as 1.3 μg/ml of psoralen could inhibit the amplification of the HVP2 DNA (FIG. 6).

In this experiment, the inhibition of PCR is less sensitive than the plaque assay. Samples treated with psoralen concentrations of 1.3 μg/ml and 2.5 μg/ml did not result in any DNA band after PCR amplification although they contained virus 20 and 4 plaques, respectively.

Inactivation of B Virus by BSPL and by the Combined Psoralen-BSPL Photo-Inactivation Technique.

Experiment 1. The Psoralen-BSPL procedure that was developed for HVP2 was applied subsequently for B virus. All procedures were similar to those described for HVP2, except that the handling of the infectious B virus was performed in the Biosafety 4 laboratory (BSL4). The B virus lab strain (E2490) (MOI=5) was grown in 95% confluent Vero cells in 850 cm² roller bottles, maintained in DMEM high glucose supplemented with 1% FBS and antibiotics. The infection progressed for 24 hr at 34° C., after which cells were scraped into the media and centrifuged at 1500 rpm (514×g) for 10 min. Cell pellets were resuspended in 2.5 ml of sterile ultrapure water. The suspension was then treated by 3 cycles of freezing on dry ice and thawing in a 37° C. water bath. Each freezing cycle lasted for at least 15 min. Cell debris was removed by centrifugation (1500 rpm/10 min) and the suspension (5 ml) containing the virus was saved. The virus titer of the suspension as determined by the plaque assay in Vero cells was approximately 10⁹ PFU/ml.

Inactivation of B virus by psoralen and BSPL was performed as described for HVP2. Briefly, two 1 ml portions of a 1:10 diluted B virus preparation (10⁸ PFU/ml) were mixed with psoralen to a final concentration of 20 μg/ml and transferred into 2 polyethylene tubings that were heat sealed at one end. The other end of each tubing was then heat-sealed at a distance of 5 cm from the first seal. Each of the tubings was irradiated with either 12 or 15 pulses of BSPL. The polyethylene tubings were placed flat on a bed of crushed ice into the Steripulse chamber at a distance of 8 shelves (4.26 inches) from the lamp window to achieve 0.45 Joules/cm² per pulse for irradiation.

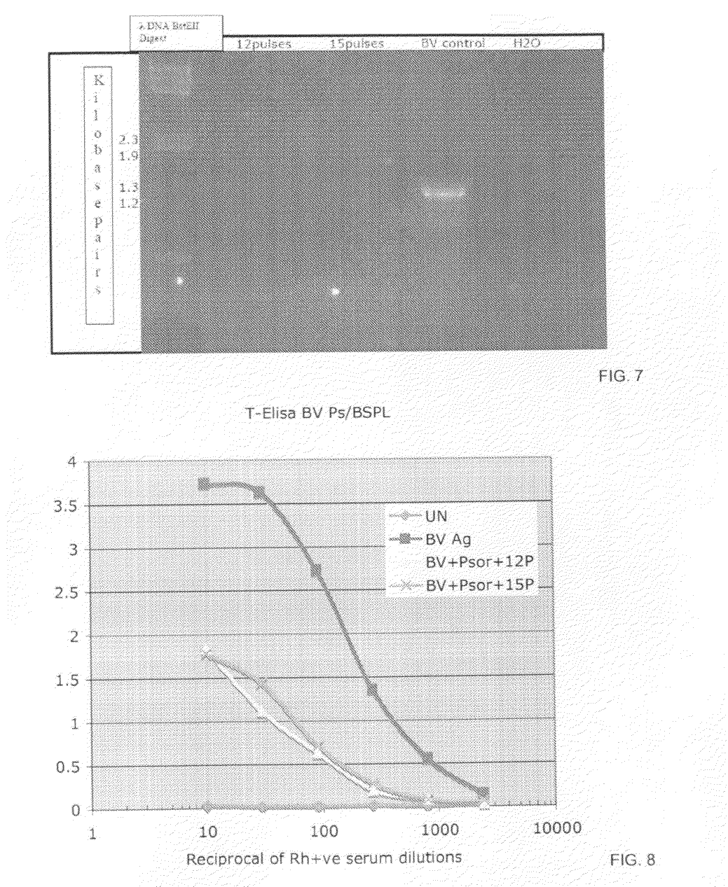

Each sample was then tested for infectivity by the plaque assay. No B virus plaques were produced after the photo-inactivation procedure with 12 or 15 BSPL pulses. The DNA damage that was caused by exposure of the B virus samples to BSPL+psoralen was also assessed by PCR. As can be seen in FIG. 7, BSPL+psoralen inhibited the PCR at both 12 and 15 BSPL pulses.

Each of the B virus photo-inactivated samples was tested for antigenicity using tELISA. A 1:30 dilution of each sample was adsorbed to microtiter plate wells, washed, blocked with Blotto and used for the titration of a rhesus anti B virus standard serum. For comparison, a standard B virus (Tween/Doc) antigen preparation (BV Ag) was also adsorbed to wells of the same plate. The BY Ag stock was prepared from a different virus than that used for the photo-inactivation experiment. Although the standard antigen resulted in higher OD values, there was no difference in antigenicity between the sample that was exposed to 12 BSPL pulses and the sample that was exposed to 15 pulses (FIG. 8).

### Preparation of an Inactivated B Virus Immunogen in Mouse Cells.

Preparation of the B virus stock mouse cells. The inactivated B virus immunogen was needed for the production of mouse monoclonal antibodies. Mouse 3T3 fibroblast cell line (developed from BALB/c) were grown in two 850 cm² roller bottles in DMEM high glucose supplemented with 10% PBS, 200 mM L-glutamine and antibiotics (penicillin and streptomycin) at 37° C. The following procedures were all performed in the BSL4 facility. Confluent cell monolayers (95%) in the roller bottles were infected with of B virus (MOI=5) (Strain E2490) and maintained in DMEM high glucose supplemented with 1% FBS and antibiotics. The infected cells were incubated for 24 hrs at 34° C., scraped into the media and centrifuged at 1500 rpm for 10 min. Cell pellets were resuspended in 4.5 ml of sterile ultrapure water. The suspension was then treated by 3 cycles of freezing on dry ice and thawing in a 37° C. water bath. Each freezing cycle lasted for at least 15 min. Cell debris was removed by centrifugation (1500 rpm/10 min) and the virus suspension (about 5 ml) was saved. The virus titer of the suspension was approximately 2×10⁷ per ml, as determined by the plaque assay in Vero cells.

The inactivation procedure. The B virus preparation (1.4 ml) was diluted 1:5 in sterile ultrapure water to a total volume of 7 ml, and 70 μl of 2 mg/ml Psoralen (4-Aminomethyl-trioxsalen hydrochloride, #A4330, Sigma) were added to the virus suspension resulting in a final concentration of 20 μg/ml. The virus-Psoralen mixture was transferred in 1 ml portions to 7 polyethylene tubings (Polyethylene (Low Density) polytubing #S-3520, 1″×1,500′, 2 Mil Poly Tubing Roll, ULINE, Atlanta Ga.) that were heat sealed at one end. After transferring the virus to the tubing the other end was heat sealed at a distance of 5 cm. The sealed tubings were decontaminated by submersion in a bottle containing CIDEX (activated glutaraldehyde solution) for 15 min. The outside of the bottle was decontaminated by submersion in the CIDEX dunk tank for 15 min. The CIDEX was removed from the bottle, and the bottle and tubings were transferred to a quaternary ammonium dunk tank through which they were removed from the BSL4 glove cabinets and then transferred to the BSL3 laboratory. The tubings were then rinsed individually with 70% alcohol. For BSPL exposure, each tubing was placed on a flat bed of ice on a tray and inserted into the irradiation chamber of the SteriPulse-XL device (RS-3000C, Xenon Corp.). The distance of the ice surface from the lamp window was 8 shelves (4.26 inches). Each of the 7 virus-containing tubings was then exposed to 12 pulses/4 seconds of BSPL that sums up to a total energy of 5.4 Joules/cm². Following irradiation the content of the individual tubings were pooled and tested for the presence of residual B virus by the plaque assay, for DNA damage by PCR, and for antigenicity by tELISA.

Plaque assay for validating the inactivation of B virus. 500 μl of the pooled B virus suspension were tested for infectivity in Vero cell monolayers grown in 6 well plates. The cultures were observed for 48 hrs. No cytopathic effect was observed. The cells from the virus infected well were then scraped and transferred to another well containing Vero cells for another 48 hours. No cytopathic effect was observed after replating. These results indicate that no live B virus could be detected after inactivation.

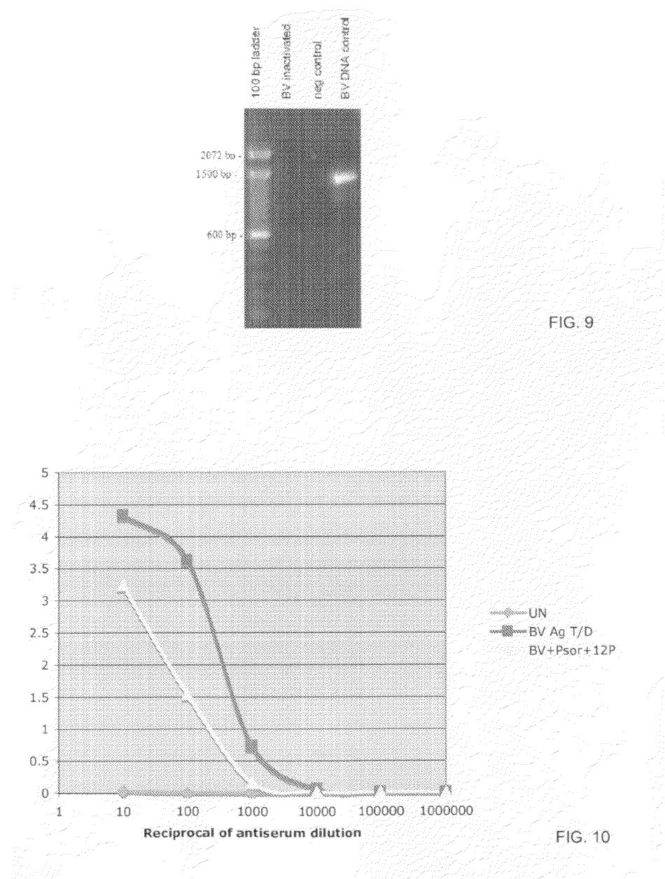

PCR results. A B virus gB primer set that amplifies a 1.3 kb fragment of the B virus genome was used. The PCR reaction was performed by using the PCR HotStar Kit (Qiagen) and 3 μl of purified DNA in 20 μl volume. The amplification was performed on ABI Thermocycler 9600 using the following cycling conditions: 15 min 95° C. and 35 two step cycles of 20 sec at 95° C. and 40 sec at 65° C. Then the PCR reaction products were run on 1% agarose gel along with the DNA marker to determine the presence of the PCR fragment of the expected size. No amplified fragment from the irradiated preparation could be demonstrated. The absence of the PCR fragment after amplification implied that DNA in the sample was damaged and could not be replicated (FIG. 9).

Antigenicity test. A 1:6 dilution of the B virus preparation was adsorbed to 96 well microtiter plates. Wells were adsorbed with a standard detergent solubilized B virus preparation (BV Ag) or mock infected cell lysates (UN). A standard rhesus anti B virus positive serum was then tested for antibodies by ELISA. Results, shown in FIG. 10, indicate that the inactivation procedure did not destroy the antigenicity of the immunogen.

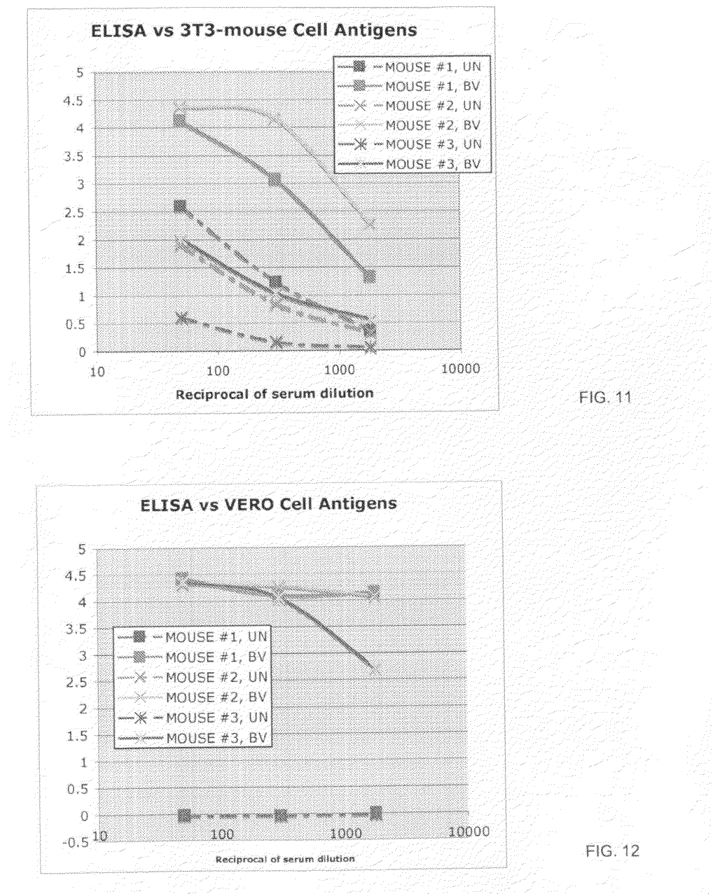

Evaluation of the immunogenicity of the inactivated B virus immunogen. The inactivated B virus immunogen was used by University of Georgia Monoclonal antibody Facility (UGA-MAF) for the preparation of mouse monoclonal antibodies. Three mice were inoculated, and bled after the second booster inoculation. The sera were tested for polyclonal antibodies by ELISA against the original immunogen that was prepared in 3T3 cells (FIG. 11) and against a standard antigen prepared in Vero cells (FIG. 12). Each set of sera were also tested against an uninfected (UN) control antigens prepared from 3T3 cells and Vero cells.

These results indicated that the immunogen prepared by the Psoralen-BSPL inactivation method induced anti B virus antibodies in all three mice. Interestingly, the immunogen that was prepared in 3T3 mouse cells induced also antibodies to the 3T3 control cells but none to the Vero cells. However, even when tested against the 3T3 cell antigens, the antibody response to B virus in all three mice was always higher than the response to the cell controls.

Conclusion A method was developed for inactivation of crude HVP2 and B virus cell extracts utilizing a combination of psoralen and Broad Spectrum Light Pulses (BSPL). A benchtop sterilization chamber from Xenon Corporation was used to generate measured BSPL. Although psoralen and “black light” (UVA) was used for inactivation of viruses for many years the combination of psoralen and BSPL is unique and was never used before to our knowledge. Previous experiments showed that 18 pulses of BSPL by itself were capable of inactivation of HVP2 infected cell extracts. These experiments demonstrate that the addition of psoralen enables virus inactivation with fewer BSPL pulses. The advantage of combining psoralen and BSPL is that the virus is inactivated by both the germicidal UV wavelength and by the photo-inactivation that is caused by the UVA (black light)-photo-activated psoralen. The damage to the DNA will therefore be greater. Another advantage of BSPL is that it is emitted in high energy short pulses (360 micro seconds) from a xenon lamp and not from mercury lamps. The relatively short exposure times are beneficial, since samples are not overheated during exposure. Validation of virus inactivation was made by infectivity assays and by PCR inhibition that is indicative of the actual photo-inactivation damage to the nucleic acids.

A B virus immunogen was prepared from a batch of B virus prepared in 3T3 mouse cells. The preparation was inactivated by using the psoralen-BSPL technique. This immunogen was used by the Monoclonal Antibody Facility at UGA for the immunization of mice for the production of monoclonal antibodies (MABs). The induction of high titers of antibodies in three of the inoculated mice indicated that immunogenicity was not impaired by the Psoralen-BSPL procedure.

### Example 2

### Herpes B Virus Immuno Dip Strip Test (BV-IDST) Kit for the Detection of Anti B Virus Antibodies in Macaque Sera

The BV-IDST is an enzyme immunoassay for the detection of herpes B virus IgG antibodies in macaque species. Each individual dip-strip is used for the detection antibodies in one blood (serum, plasma or whole blood) sample. The test is simple, and requires little if any laboratory equipment and therefore suitable for field-testing.

Introduction. Herpes B virus (Herpesvirus simiae or Cercopithecine herpesvirus 1), a member of the Alphaherpesvirinae subfamily and the Simplexvirus group, is known to occur naturally in macaques (Macaca spp). Infection of macaques may be asymptomatic or may cause a mild disease. Infection of other species (including humans) is rare but results in severe, and often, if untreated, lethal disease.

Past infections are determined by detection of anti B virus antibodies using serological assays. Serological diagnosis of B virus infections in humans is complicated by the relatively high prevalence of the immunologically cross-reacting herpes simplex virus infections (HSV-1 and/or HSV-2). Past infections in macaques can be established without these complications because the only simplexvirus known to infect macaques is B virus. Identifying B virus infected macaques is important for managing macaques in captivity, for developing specific pathogen free colonies and for the prevention of the potential exposure and infection of humans who handle macaques.

The virus antigen for these assays is prepared from detergent solubilized and inactivated B virus infected cells (BV) and the negative antigen control is prepared in a similar way from uninfected cell lysates (UN). The antigen can also be prepared using the compositions and methods described in Example 1.

The BV-IDST was developed to enable field-testing to detect B virus antibodies in macaque sera. The principle of the BV-IDST is similar to the principle of ELISA except that nitrocellulose strips are used instead of plastic wells as the solid phase to which antigens are adsorbed. No special laboratory equipment (washers, readers, etc.) is necessary for carrying out the test.

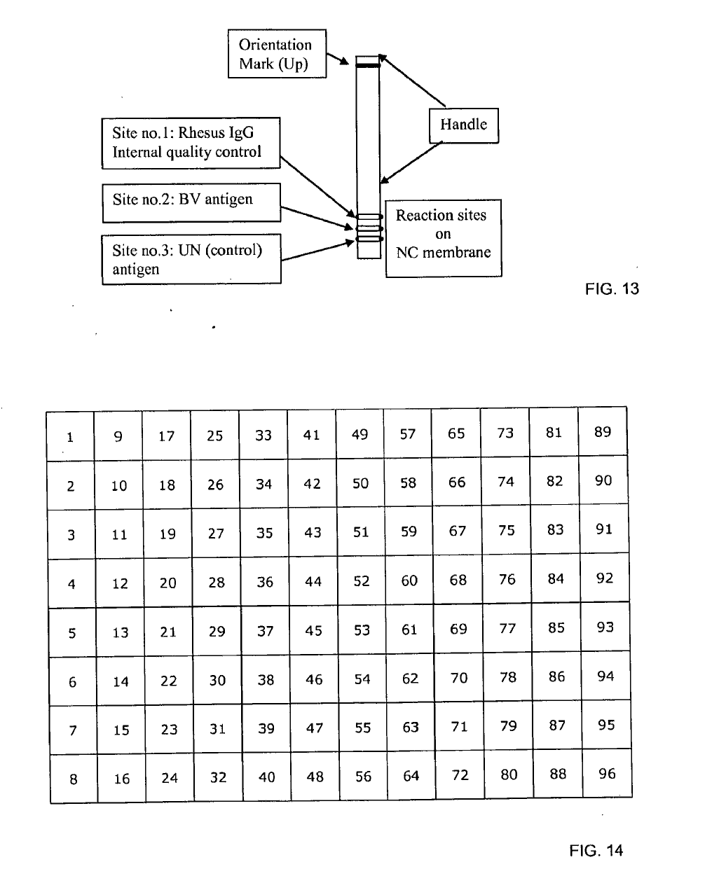

For example, one BV-IDST kit can include about 100 individual nitrocellulose strips on which control and B virus antigens are pre-applied to predetermined reaction sites. Each strip contains 3 reaction sites as shown in FIG. 13. Site #1 serves as an internal quality control for the anti human IgG conjugate; it contains normal rhesus IgG. The IgG control also serves as a reference line for reading the results since, in a properly developed test, it will always be visible. Site #2 includes the BV antigen, and site #3 includes the uninfected control antigen (UN). In one embodiment, the BV and UN antigens are detergent solubilized cell lysates prepared as described for the conventional ELISA. (See, Katz, D., W. Shi, M. Wildes, and J. K. Hilliard. 2002. Automation of serological diagnosis of herpes B virus infections using robot-assisted integrated workstations. JALA 7: 110-115). The BV-IDST is performed by dipping each strip sequentially into the diluted test sample (30 min), into the conjugate (30 min), and into the insoluble chromogenic substrate (5-10 min), with intermediate short tap-water rinsing procedures. The BV-IDST can be used for detecting the presence of antibodies in serum, plasma or whole blood. Positive and negative serum controls (provided) are also tested along with the unknown samples. The test is completed in approximately 80 minutes. Results are read by eye.

In one embodiment of the a BV-IDST kit, the following materials can be provided to perform 100 antibody tests:
1. A total of 100 IDS strips packaged in 4 sealed plastic bags (25 strips per bag).
2. One (empty) 96 deep well box for preparing the dilutions of the tested serum samples. The 96 deep well box can be reused after soaking in dish washer detergent and thorough rinsing in tap water.
3. Two conical tubes, each containing 50 ml dilution buffer (PBS+Az) for diluting blood samples and for the dilution of the conjugate.
4. Eight incubation plastic trays.
5. One tube containing 0.200 ml of goat anti-human IgG conjugated with alkaline phosphatase.
6. One tube containing 0.250 ml negative control serum.
7. One tube containing 0.250 ml positive control serum.
8. 20 ml ready to use substrate (NBT/BCIP) in four 50 ml conical tubes.

The BV-IDS Test can be preformed utilizing the following procedure:
1. Prepare a list of the samples to be tested. Assign a serial number to each serum. Label the strips with serial numbers that correspond to the number of sera tested. The location of the wells in the 96 deep well box that correspond to the strip numbers are suggested in FIG. 14 as follows: start with number 1 in the upper left corner well, and count down the column ending with well number 8. The second column of wells will accommodate sera numbered from 9 to 16, the third will accommodate sera numbered from 17 to 24 and so on (See FIG. 14). The strips are marked with numbers 1 to 96. A maximum of 94 test samples and 2 control sera (Negative and Positive) can be tested in one 96 deep well box.
2. The following instructions and measures are for testing 96 strips at the same time. If less strips are tested, the relative amount of buffer and reagents to be used should be calculated.
3. Mark the left upper corner of the 96-deep well box for future orientation. Fill 0.5 ml of the PBS+Az dilution buffer into the number of wells that correspond to the number of samples that will be tested+2 additional wells for the negative and positive control sera.
4. Dilute test and control sera 1:20 by adding 0.025 ml (25 μl) of each serum sample to each of the buffer-filled wells.
5. Remove the appropriate number of strips from the plastic bag or bags. Using a permanent ink felt tip marker, mark each strip with a number that will correspond to the serum number to be tested. Observe the serial number on the “handle” of the strip and dip it in the corresponding well according to the matrix shown in FIG. 14.
6. Incubate for 30 min at room temperature (approximately 25° C. or 77° F.).
7. While the strips are incubating, prepare the conjugate dilution 1:1000 by adding 0.05 ml to the 50 ml PBS in the conical tube. Mix well.
8. Remove each strip from the serum incubation box and rinse in tap water. Rinse each strip at a time and place the strip face up into the conjugate tray or trays. Each plastic tray can accommodate 25 to 27 IDS strips. If all 96 strips are tested, you will need 4 trays.
9. Using a Pasteur pipette (or any other pipette) apply the diluted conjugate over the nitrocellulose part. To prevent the strips from floating, use a volume that will be just enough to cover the nitrocellulose part (the test end) of the strip. About 5 ml of the conjugate may be needed to cover 27 strips. Be sure that the reactive area of the strips are covered with the conjugate solution.
10. Incubate in the strips in the conjugate tray for 30 min at room temperature (25° C. or 77° F.).
11. At the end of the conjugate incubation period rinse the strips in tap water as before and place them (face up) into the substrate plastic tray or trays.
12. Add the substrate solution on top of the strips as described in paragraph no. 9.
13. Develop in the substrate solution for 5-10 min. S top developing when the expected lines appear on strips that were incubated with the negative and positive control sera.
14. Rinse with tap water as before placing the strips on dry filter paper. Read results after the strips are totally dry.

Interpretation of test results. Ideally, strips that are incubated with the negative serum control and negative samples will show only one blue colored band at reaction site no. 1 (FIG. 15A). Strips that are incubated with the positive control serum and with positive samples will show two blue colored bands at reaction sites no. 1 and no. 2 (FIG. 15B). However, in some cases a band may appear at reaction site no. 3. In these cases compare the band in site no. 2 to the band in site no. 3. If intensity is similar, fail the test because this indicates a background reaction. If band at site 2 is stronger than the band in site 3, the result is positive.

If the outcome of the positive and negative control sera is not as expected the whole test should be failed. If the results of the negative and positive control sera are as expected but the band at reaction site #1 of one of the test strips does not appear with a particular test sample, the test should be failed for this sample only. Failed tests should be repeated.

Preparation of nitrocellulose IDS (Immuno Dip Strips). G&L, Precision Die Cutting Inc. cards were used to back nitrocellulose (NC) membranes. Osmonic Inc., NitroPure, Supported Nitrocellulose, 0.45μ, Cat. No. WP4HY417F2, Material No. 1214935 were used. (The same membranes are used for WB by our Dx Lab, custom cut to 14×16 cm, ordered from Fisher, Cat. No. 9910523)

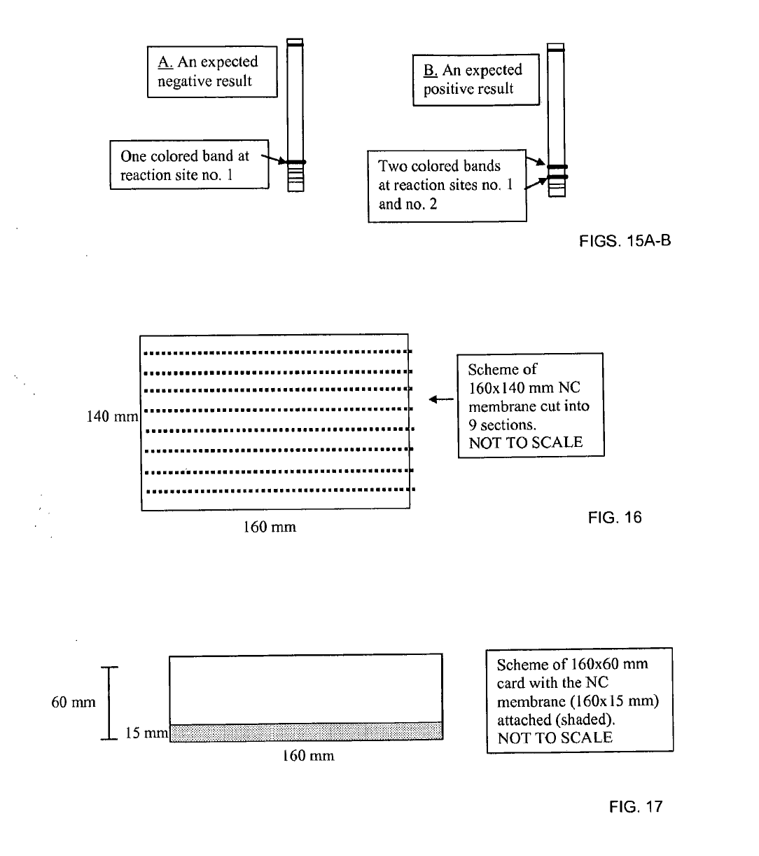

Cut each membrane sheet, using a sharp “exacto” knife in 160 mm×15 mm strips. Nine strips of NC, each measuring 160×15 mm, can be cut from one 140×160 mm membrane. Peel the 15 mm section on the backing card and apply the NC membrane on the sticky surface. (see FIGS. 16-17).

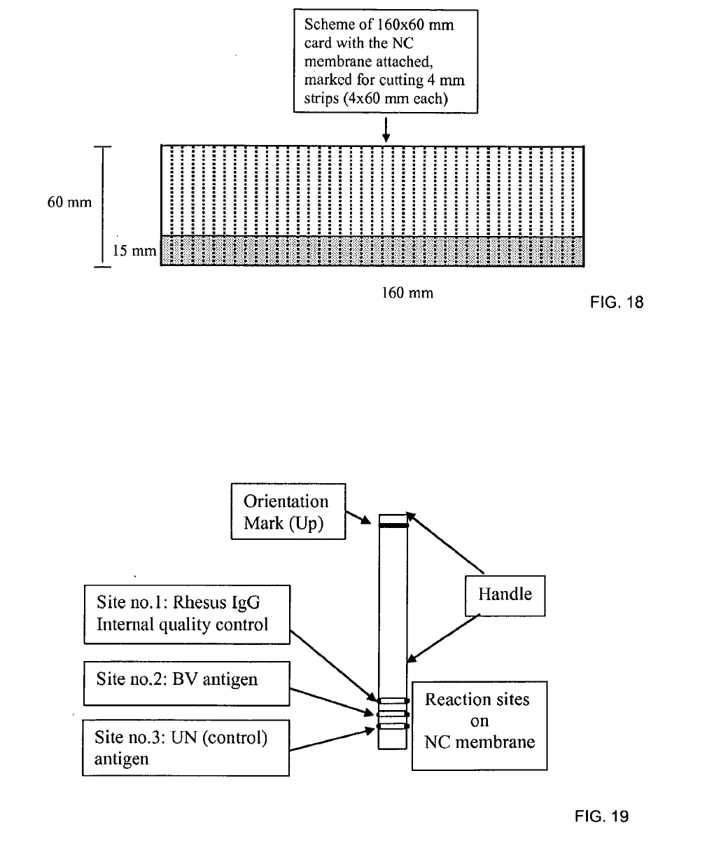

Each 160×60 card, with its attached NC membrane can be cut in 40 (60×4 mm) strips for producing 40 IDS strips (FIG. 18). Antigens can be applied to the nitrocellulose card or strips. For example, 3 antigen lines can be sprayed on the nitrocellulose section using the BioDot AD 1500 (Program: “Line dispense 1-17-08.ad*-BioDot Ax Sys”).

### Example 3

### Herpes B Virus Immuno Dip Strip Test (BV-IDST) Kit for the Detection of Anti B Virus Antibodies in Macaque Sera

The BV-IDST is an enzyme immunoassay for the detection of herpes B virus IgG antibodies in macaque species. Each individual dip-strip is used for the detection antibodies in one blood (serum, plasma or whole blood) sample. The test is simple, and requires little if any laboratory equipment and therefore suitable for field-testing.

Introduction. Herpes B virus (Herpesvirus simiae or Cercopithecine herpesvirus 1), a member of the Alphaherpesvirinae subfamily and the Simplexvirus group, is known to occur naturally in macaques (Macaca spp). Infection of macaques may be asymptomatic or may cause a mild disease. Infection of other species (including humans) is rare but results in severe, and often, if untreated, lethal disease.

Past infections are determined by detection of anti B virus antibodies using serological assays. Serological diagnosis of B virus infections in humans is complicated by the relatively high prevalence of the immunologically cross-reacting herpes simplex virus infections (HSV-1 and/or HSV-2). Past infections in macaques can be established without these complications because the only simplexvirus known to infect macaques is B virus. Identifying B virus infected macaques is important for managing macaques in captivity, for developing specific pathogen free colonies and for the prevention of the potential exposure and infection of humans who handle macaques.

The virus antigen for these assays is prepared from psoralen/BSPL inactivated B virus infected cell lystates (BV), and the negative antigen control is prepared in a similar way from uninfected cell lysates (UN).

The BV-IDST was developed to enable field-testing to detect B virus antibodies in macaque sera. The principle of the BV-IDST is similar to the principle of ELISA except that nitrocellulose strips are used instead of plastic wells as the solid phase to which antigens are adsorbed. No special laboratory equipment (washers, readers, etc.) is necessary for carrying out the test.

For example, one BV-IDST kit can include about 100 individual nitrocellulose strips on which control and B virus antigens are pre-applied to predetermined reaction sites. Each strip contains 3 reaction sites as shown in FIG. 13. Site #1 serves as an internal quality control for the anti human IgG conjugate; it contains normal rhesus IgG. The IgG control also serves as a reference line for reading the results since, in a properly developed test, it will always be visible. Site #2 includes the BV antigen, and site #3 includes the uninfected control antigen (UN). In one embodiment, the BV and UN antigens are psoralen/BSPL inactivated cell lysates. The By-IDST is performed by dipping each strip sequentially into the diluted test sample (30 min), into the conjugate (30 min), and into the insoluble chromogenic substrate (5-10 min), with intermediate short tap-water rinsing procedures. The BV-IDST can be used for detecting the presence of antibodies in serum, plasma or whole blood. Positive and negative serum controls (provided) are also tested along with the unknown samples. The test is completed in approximately 80 minutes. Results are read by eye.

In one embodiment of a BV-IDST kit, the following materials can be provided to perform 100 antibody tests:
1. A total of 100 IDS strips packaged in 4 sealed plastic bags (25 strips per bag).
2. One (empty) 96 deep well box for preparing the dilutions of the tested serum samples. The 96 deep well box can be reused after soaking in dish washer detergent and thorough rinsing in tap water.
3. Two conical tubes, each containing 50 ml dilution buffer (PBS+Az) for diluting blood samples and for the dilution of the conjugate.
4. Eight incubation plastic trays.
5. One tube containing 0.200 ml of goat anti-human IgG conjugated with alkaline phosphatase.
6. One tube containing 0.250 ml negative control serum.
7. One tube containing 0.250 ml positive control serum.
8. 20 ml ready to use substrate (NBT/BCIP) in four 50 ml conical tubes.

The BV-IDS Test can be preformed utilizing the following procedure:
9. Prepare a list of the samples to be tested. Assign a serial number to each serum. Label the strips with serial numbers that correspond to the number of sera tested. The location of the wells in the 96 deep well box that correspond to the strip numbers are suggested in FIG. 14 as follows: start with number 1 in the upper left corner well, and count down the column ending with well number 8. The second column of wells will accommodate sera numbered from 9 to 16, the third will accommodate sera numbered from 17 to 24 and so on (See FIG. 14). The strips are marked with numbers 1 to 96. A maximum of 94 test samples and 2 control sera (Negative and Positive) can be tested in one 96 deep well box.
10. The following instructions and measures are for testing 96 strips at the same time. If less strips are tested, the relative amount of buffer and reagents to be used should be calculated.
11. Mark the left upper corner of the 96-deep well box for future orientation. Fill 0.5 ml of the PBS+Az dilution buffer into the number of wells that correspond to the number of samples that will be tested +2 additional wells for the negative and positive control sera.
12. Dilute test and control sera 1:20 by adding 0.025 ml (25 μl) of each serum sample to each of the buffer-filled wells.
13. Remove the appropriate number of strips from the plastic bag or bags. Using a permanent ink felt tip marker, mark each strip with a number that will correspond to the serum number to be tested. Observe the serial number on the “handle” of the strip and dip it in the corresponding well according to the matrix shown in FIG. 14.
14. Incubate for 30 min at room temperature (approximately 25° C. or 77° F.).
15. While the strips are incubating, prepare the conjugate dilution 1:1000 by adding 0.05 ml to the 50 ml PBS in the conical tube. Mix well.
16. Remove each strip from the serum incubation box and rinse in tap water. Rinse each strip at a time and place the strip face up into the conjugate tray or trays. Each plastic tray can accommodate 25 to 27 IDS strips. If all 96 strips are tested, 4 trays are used.
17. Using a Pasteur pipette (or any other pipette) apply the diluted conjugate over the nitrocellulose part. To prevent the strips from floating, use a volume that will be just enough to cover the nitrocellulose part (the test end) of the strip. About 5 ml of the conjugate may be needed to cover 27 strips. Be sure that the reactive area of the strips are covered with the conjugate solution.
18. Incubate in the strips in the conjugate tray for 30 min at room temperature (25° C. or 77° F.).
19. At the end of the conjugate incubation period rinse the strips in tap water as before and place them (face up) into the substrate plastic tray or trays.
20. Add the substrate solution on top of the strips as described in paragraph no. 9.
21. Develop in the substrate solution for 5-10 min. S top developing when the expected lines appear on strips that were incubated with the negative and positive control sera.
22. Rinse with tap water as before placing the strips on dry filter paper. Read results after the strips are totally dry.

Interpretation of test results. Ideally, strips that are incubated with the negative serum control and negative samples will show only one blue colored band at reaction site no. 1 (FIG. 15A). Strips that are incubated with the positive control serum and with positive samples will show two blue colored bands at reaction sites no. 1 and no. 2 (FIG. 15B). However, in some cases a band may appear at reaction site no. 3. In these cases compare the band in site no. 2 to the band in site no. 3. If intensity is similar, fail the test because this indicates a background reaction. If band at site 2 is stronger than the band in site 3, the result is positive.

If the outcome of the positive and negative control sera is not as expected the whole test should be failed. If the results of the negative and positive control sera are as expected but the band at reaction site #1 of one of the test strips does not appear with a particular test sample, the test should be failed for this sample only. Failed tests should be repeated.

Preparation of nitrocellulose IDS (Immuno Dip Strips). G&L, Precision Die Cutting Inc. cards were used to back nitrocellulose (NC) membranes. Osmonic Inc., NitroPure, Supported Nitrocellulose, 0.45μ, Cat. No. WP4HY417F2, Material No. 1214935 were used. (The same membranes are used for WB by our Dx Lab, custom cut to 14×16 cm, ordered from Fisher, Cat. No. 9910523)

Cut each membrane sheet, using a sharp “exacto” knife in 160 mm×15 mm strips. Nine strips of NC, each measuring 160×15 mm, can be cut from one 140×160 mm membrane. Peel the 15 mm section on the backing card and apply the NC membrane on the sticky surface. (see FIGS. 16-17)

Each 160×60 card, with its attached NC membrane can be cut in 40 (60×4 mm) strips for producing 40 IDS strips. (FIG. 18). Antigens can be applied to the nitrocellulose card or strips. For example, 3 antigen lines can be sprayed on the nitrocellulose section using the BioDot AD 1500 (Program: “Line dispense 1-17-08.ad*-BioDot Ax Sys”).

### REFERENCES

1. Lin L., Hanson C. V., Alter H. J., Jauviin, V., Bernard K. A., Murthy K. K., Metzel P. and Corash L. Inactivation of viruses in platelet concentrates by photochemical treatment with amotosalen and long-wavelength ultraviolet light. Transfusion, 45, 580-590, 2005.
2. Couvet-Privat S., Mace G., Rosseli F., and Saparbaev M. K. Psoralen-induced DNA adducts are substrates for base excision repair pathway in human cells. Nucleic Acids Research, 35, 5672-5682, 2007.
3. Aytay S., Ohagen A., Busch M. R., Alford B. Chapman J. R. and Lazo A. Development of a sensitive PCR inhibition method to demonstrate HBV nucleic acid inactivation. Transfusion, 44, 476-484, 2004.
4. Allain J-P., Hsu J., Pranmeth M., Hanson D., Stassinopoulos A., Fischetti L., Corash L., and Lin L. Quantification of viral inactivation by photochemical treatment with amotosalen and UV A light, using a novel polymerase chain reaction inhibition method with preamplification. The Journal of Infectious Diseases, 194, 1737-1744, 2006.
5. Lin L., Londe H., Hanson C. V., Wiesehahn G. P., Cimino G. D., and Corash L. Validation of psoralen photochemical inactivation of HIV-1 in platelet concentrates using PCR inhibition or Rt/PCR inhibition assay. Int. Conf. AIDS (Abstract no. PO-B42-2491), 9, 550, Jun. 6-11, 1993.
6. Lin L., Dikeman R., Molini B., Lukehart S. A., Lane R., Dupuis K., Metzel P., and Corash L. Photochemical treatment of platelet concentrates with amotosalen and long-wavelength ultraviolet light inactivates a broad spectrum of pathogenic bacteria. Transfusion, 44, 1496-1504, 2004.
7. Jansen G. A. J., Van Vliet H. H. D. M., Vermeij H., Beckers E. A. M., Leebeek F. W. G., Sonneveld P., and van Rhenen D. J. Functional characteristics of photochemically treated platelets. Transfusion, 44, 313-319, 2004.
8. Katz D, Hilliard J K, Eberle R, Lipper S L (1986a) ELISA for detection of group-common and virus-specific antibodies in human and simian sera induced by herpes simplex and related simian viruses. J Virol Methods 14: 99-109
9. Eberle, R., and J. Hilliard. 1995. The simian herpesviruses. Infect. Agents Dis. 4: 55-70.
10. Katz, D., W. Shi, P. W. Krug, R. Henkel, H. McClure and J. K. Hilliard. 2002. Antibody cross reactivity of alphaherpesviruses as mirrored in naturally infected primates. Arch. Virol. 147: 929-941.
11. Katz, D., W. Shi, M. Wildes, and J. K. Hilliard. 2002. Automation of serological diagnosis of herpes B virus infections using robot-assisted integrated workstations. JALA 7: 110-115.

1. A method for inactivating a microorganism, comprising:
providing at least one furanocoumarin to a microorganism; and
exposing the microorganism to at least one pulse of a broad spectrum pulsed light, thereby inactivating the microorganism;
wherein exposing the microorganism to at least one pulse of a broad spectrum pulsed light comprises exposing the microorganism to about 0.45 Joule/cm² to about 13.5 Joules/cm² of broad spectrum light.

2. The method of claim 1, wherein the microorganism is selected from the group consisting of viruses, bacteria, and fungi.

3. The method of claim 1, wherein the microorganism is a virus.

4. The method of claim 3, wherein the virus is a herpesvirus.

5. The method of claim 4, wherein the herpesvirus is selected from the group consisting of herpes B virus and herpes virus papio 2.

6. The method of claim 1, wherein the at least one furanocoumarin comprises a psoralen.

7. The method of claim 6, wherein psoralen is present in a concentration of about 0.1 μg/ml to about 60 μg/ml.

8. The method of claim 7, wherein psoralen is present in a concentration of at least about 5 μg/ml.

9. (canceled)

10. The method of claim 1, wherein exposing the microorganism to at least one pulse of a broad spectrum pulsed light comprises exposing the microorganism to at least about 4.05 Joules/cm² of broad spectrum light to about 13.5 Joules/cm² of broad spectrum light.

11. An inactivated microorganism, comprising a photo-chemically inactivated nucleic acid, wherein the photo-chemically inactivated nucleic acid is photo-chemically inactivated by at least one furanocoumarin and at least one pulse of a broad spectrum pulsed light;
wherein the at least one pulse of a broad spectrum pulsed light comprises about 0.45 Joule/cm² to about 13.5 Joules/cm² of broad spectrum light.

12. The inactivated microorganism of claim 11, wherein the microorganism is selected from the group consisting of viruses, bacteria, and fungi.

13. The inactivated microorganism of claim 11, wherein the microorganism is a virus.

14. The inactivated microorganism of claim 13, wherein the virus is a herpesvirus.

15. The inactivated microorganism of claim 14, wherein the herpesvirus is selected from the group consisting of herpes B virus and herpes virus papio 2.

16. The inactivated microorganism of claim 11, wherein the at least one furanocoumarin comprises a psoralen.

17. The inactivated microorganism of claim 16, wherein the psoralen is present in a concentration of about 0.1 μg/ml to about 60 μg/ml.

18. (canceled)

19. The inactivated microorganism of claim 15, wherein the at least one furanocoumarin comprises psoralen at a concentration of at least about 5 μg/ml, and wherein the at least one pulse of a broad spectrum pulsed light comprises at least about 4.05 Joules/cm² of broad spectrum light.

20-56. (canceled)
